(19) United States
(12) Patent Application Publication
(10) Pub. No.: US 2016/0074632 A1
(43) Pub. Date: Mar. 17, 2016

(54) BALLOON CATHETER WITH UNCOATED BALLOON PORTION OR SECOND UNCOATED BALLOON

(71) Applicant: BIOTRONIK AG, Buelach (CH)

(72) Inventors: Raimund Moehl, Forch (CH); Michael Filippi, Zuerich (CH); Andreas Hofmann, Graefenberg (DE); Bodo Quint, Dettighofen (DE)

(21) Appl. No.: 14/824,257

(22) Filed: Aug. 12, 2015

Related U.S. Application Data
(60) Provisional application No. 62/050,212, filed on Sep. 15, 2014.
(60) Provisional application No. 62/050,211, filed on Sep. 15, 2014.

(51) Int. Cl. A61M 25/10; A61L 29/16; A61L 29/06

(52) U.S. Cl. CPC A61M 25/10; A61L 29/06; A61L 29/16; A61L 2300/606

(57) ABSTRACT

A balloon catheter includes a drug-coated balloon having an uncoated region free from drug coating, a displaceable protective sleeve covering the drug-coated balloon and an elongate catheter body that has a distal end and a proximal end. Another catheter has an uncoated region and/or an additional uncoated balloon is on the catheter body. The uncoated balloon or the additional uncoated balloon are mounted to be exposed first when the sleeve is displaced. This blocks blood flow while the drug coated balloon or balloon region is inflated to avoid or minimize release of drug into the blood stream away from the site of treatment.

### PRIORITY CLAIM

This application claims priority under 35 U.S.C. §119 from prior U.S. Provisional Application No. 62/050,212, filed Sep. 15, 2014 and from U.S. Provisional Application No. 62/050,211, filed Sep. 15, 2014.

### FIELD OF THE INVENTION

A field of the invention is balloon catheter devices, particularly devices including a drug-coated balloon.

### BACKGROUND

Balloons are a key functional element of balloon catheters, as are used (inter alia) to dilate constricted vessels and to introduce and expand stents in vessels or hollow organs. Reliable function thereof is of utmost importance for the success of corresponding interventions.

It has been attempted for some time to increase the sustainability of the dilation, performed by balloon catheters, of constricted vessels and to improve the overall performance of such balloon catheters by applying a suitable drug coating to the balloon. Balloons having a coating of this type, which are also referred to as DEBs (drug-eluting balloons), are prior art and are in clinical use.

Known drug-coated balloons have a disadvantage concerning drug release on the way to the treatment site. This means that the amount of active ingredient at the treatment site varies and that the detached particles may be deposited at other sites in an undefined manner. The effect of the drug coating is thus difficult to control to a certain degree.

### SUMMARY OF THE INVENTION

A balloon catheter includes a drug-coated balloon having an uncoated region free from drug coating, a displaceable protective sleeve covering the drug-coated balloon and an elongate catheter body that has a distal end and a proximal end. The catheter blocks blood flow while the drug coated balloon or balloon region is inflated to avoid or minimize release of drug into the blood stream away from the site of treatment. In another balloon catheter, blood flow is blocked by an uncoated balloon.

### BRIEF DESCRIPTION OF THE DRAWINGS

Advantages and expedient features of the invention will also become clear from the following description of exemplary embodiments on the basis of the figures, in which:

FIGS. 1A to 1D schematic view of a first embodiment of the invention,

FIGS. 2A to 2C schematic view of a second embodiment of the invention, and

FIGS. 3A to 3C schematic views of additional embodiments of the invention.

### DETAILED DESCRIPTION OF THE PREFERRED EMBODIMENTS

Preferred embodiments provide a balloon catheter with a drug coated balloon having uncoated (drug free) portion or a drug coated balloon and an uncoated balloon. The coating of the balloon is provided with a removable protection in the form of a displaceable sleeve. The protection protects the drug coating until the balloon is moved to the treatment site with the catheter. The balloon is configured so that blood flow can be interrupted until the particles that may have formed during inflation of the balloon are embedded in the vessel wall as a result of the balloon pressure.

In a preferred embodiment the balloon is divided into a coated part and an uncoated part. In a preferred embodiment, a separate coated balloon and an uncoated balloon are assembled on the inner shaft of a catheter. The uncoated balloon or uncoated part of a balloon closes the vessel distally and/or proximally and prevents drug particles from being washed away. When using separate balloons, the uncoated balloon may also be used for pre-dilation of the vessel.

In a preferred embodiment, the balloon catheter has an individual balloon that has an uncoated distal portion and a coated portion, which have the same diameter, with a common lumen so that the uncoated portion is dilated with the same pressure as the coated portion. In a variation of this embodiment, the uncoated portion has a smaller diameter than the coated portion or tapers toward its end.

In another preferred embodiment, the balloon catheter has two balloons, an uncoated balloon and a coated balloon, with the same diameter, and there are two lumens. The uncoated distal balloon can be dilated with a lower pressure than the coated balloon.

In a further preferred embodiment the balloon catheter has two balloons, an uncoated balloon and a coated balloon, wherein the distal uncoated balloon has a smaller diameter than the coated balloon, and one lumen is provided so that the uncoated distal balloon is dilated with the same pressure as the coated balloon.

The coated balloon region or coated balloon is protected by a retractable protective sleeve. This can be retracted automatically or withdrawn manually.

Compared to known balloon catheters including a drug-coated balloon, considerable clinical advantages can be achieved with the invention. Preferred embodiments provide for no, or at least minor, particle loss on the way to the stenosis and during dilation, and the distribution of active ingredient at the target site can be more predictable and homogeneous. This makes it possible to implement smaller doses, with the additional advantage of a lesser systemic delivery of active ingredient. The clear clinical advantages lead to equally clear improvements in the product properties.

In a further embodiment of the invention, the position of the drug-coated balloon or of the drug-coated region thereof, in particular an end thereof, on the catheter body is marked by at least one X-ray marker. In particular, the ends of the drug-coated balloon and the borders between the drug-coated region and the, or each, drug-free region or the ends of the drug-coated balloon and the ends of the, or each, drug-free region are each marked by an X-ray marker.

In a preferred embodiment in which the (during use) distal end of the balloon or an additional distal balloon is free from a drug coating, the protective sleeve can be withdrawn conventionally from distally to proximally by proximally grasping the protective sleeve. In a preferred embodiment with a proximally arranged drug-free region or balloon, protective sleeve can be withdrawn from proximally to distally. A displacement structure for displacing the protective sleeve toward the distal end to expose the drug-coated balloon or drug-coated region are provided in this embodiment in the catheter body, arranged in such a way that the proximally arranged drug-free balloon or portion is exposed before exposure of the drug-coated balloon or drug-coated region. Preferred displacement structures include a push-in wire or a push rod, which reaches as far as the proximal end of the catheter body, where it is provided with an actuation device.

In a further embodiment, the end of the protective sleeve facing the drug-free balloon or drug-free region can be expanded in a flexible manner. This tip protects the coating when the coated balloon is opened until it bears against the vessel wall. The flexible material of the tip has no disadvantage after the intervention during removal of the catheter.

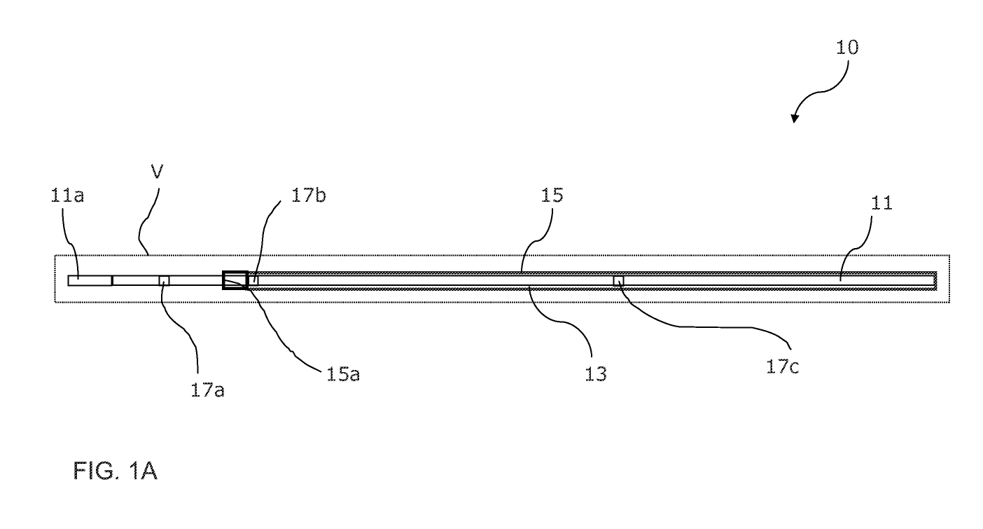

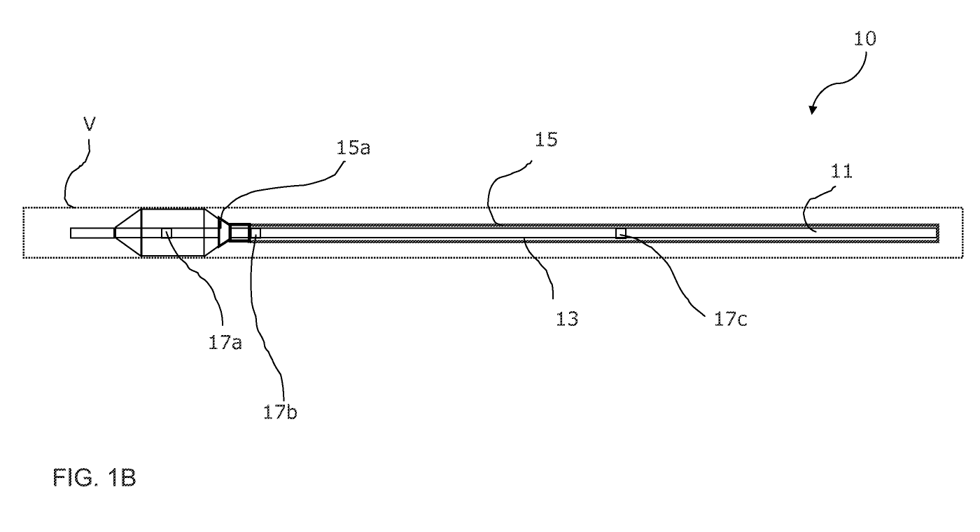

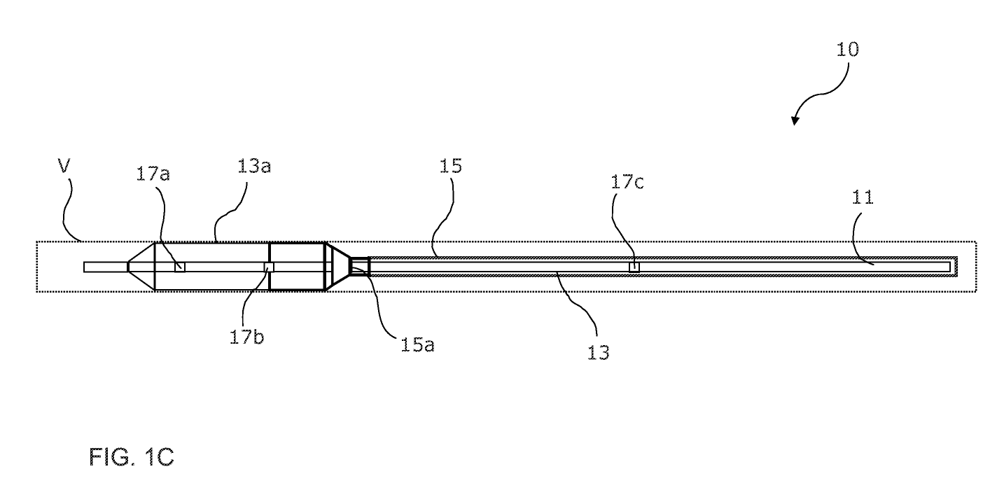

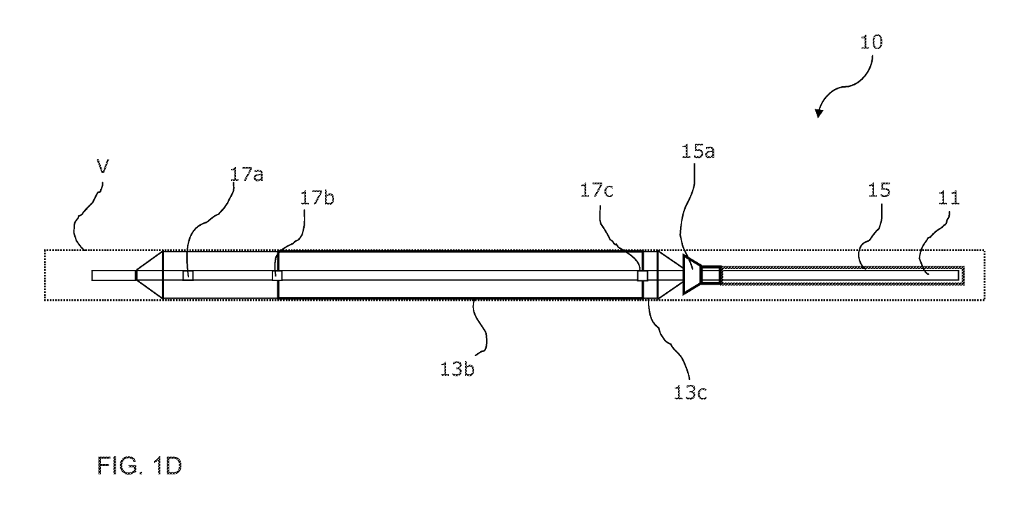

FIG. 1A to 1D each show schematic views of a phase of the insertion of a balloon catheter 10 according to the invention in a vessel V of a human (or animal) body. The balloon catheter 10 comprises an elongate catheter body 11 with a distal end 11a and a proximal end, which lies outside the body and therefore outside the region illustrated in the drawing. A balloon 13 is attached to the catheter body 11 and is folded together in the starting state (FIG. 1A) and is arranged beneath a displaceable protective sleeve 15. This sleeve, at its distal end, has an atraumatic tip 15a that can be expanded in a flexible manner. X-ray marker rings 17a, 17b and 17c are provided to identify specific portions of the balloon (see further below).

Whereas, in FIG. 1A, the state of the balloon catheter immediately after insertion at the site of use is shown, that is to say with the original position of the protective sleeve 15, FIGS. 1B to 1D show various phases of the retraction of the protective sleeve and therefore the accompanying expansion of the balloon. In FIG. 1B the protective sleeve 15 is retracted by a relatively small amount and exposes a small distal region of the balloon 13. It can also be seen in this instance that the tip 15a of the protective sleeve also expands during retraction and simultaneous expansion of the balloon. In the state according to FIG. 1C, the balloon is expanded over a greater part of its length and the protective sleeve is accordingly retracted further.

It is also shown in this instance that a distal portion 13a of the balloon is bordered by a proximal portion 13b (labelled in FIG. 1D), and more specifically the distal portion 13a is free from a drug coating. The border between the uncoated distal balloon portion 13a and the coated proximal portion 13b is also marked for X-ray imaging methods by an X-ray marker ring 17b. The tip 15a is expanded to a greater extent in this instance than in the state shown in FIG. 1B. In this illustration it can also be seen that the X-ray marker ring 17c marks the proximal end of the coated balloon portion 13b. This is adjoined further proximally by a short uncoated balloon portion 13c (shown in FIG. 1D), which is followed by the proximal balloon cone (not denoted separately).

FIG. 1D shows the fully expanded state of the balloon 13 with the protective sleeve 15 retracted to a correspondingly far extent. The tip 15a of the protective sleeve is again illustrated in the expanded state in this instance, but collapses again in this state of the balloon catheter, whereby injuries to the vessel when the catheter is withdrawn are avoided.

It can be seen in the procedure illustrated in FIGS. 1A to 1D that the vessel is initially closed by the distal uncoated balloon portion 13a before the proximal balloon portion 13b is expanded and the drug coating in this portion is thus exposed. Transport of particles from the drug coating into the blood stream and away from the site of use is thus prevented or minimal. Instead, the drug is incorporated into the vessel wall when the balloon portion 13b is fully expanded, before the balloon is deflated again and the balloon catheter is withdrawn.

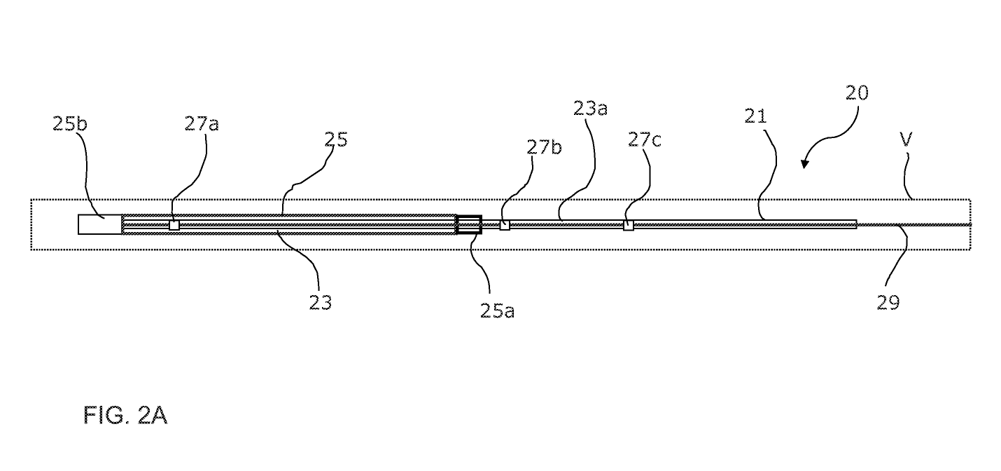

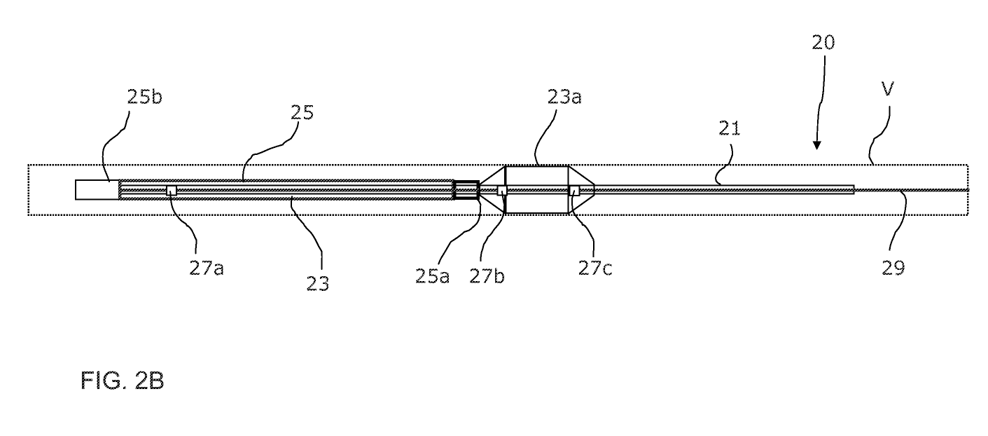

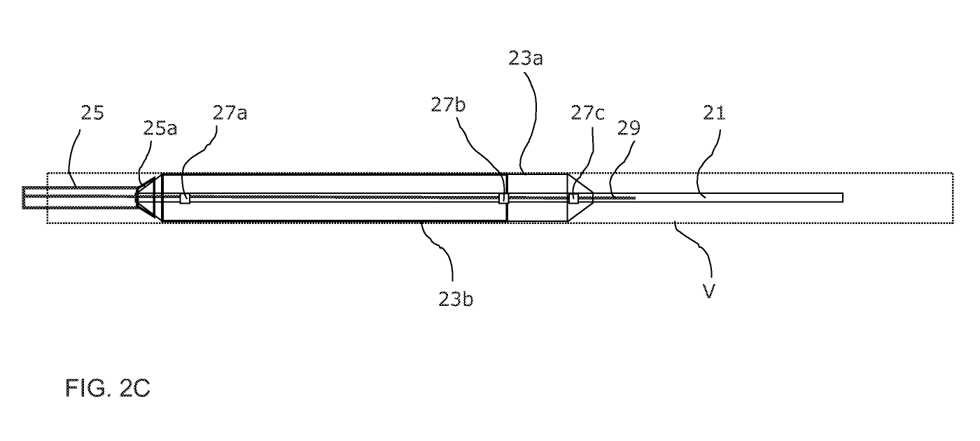

As a further embodiment of the invention, FIGS. 2A to 2C show a balloon catheter 20, of which the components are provided with common reference numbers based on the above-described embodiment shown in FIGS. 1A to 1D and will only be described again in this instance insofar as is necessary to explain the deviating features of this embodiment. A main difference lies in the fact that, in this case, the proximal end portion 23a of the balloon 23 is drug-free, thus resulting in the further basic difference that the protective sleeve 25 is not displaceable from distally to proximally, but from proximally to distally. The uncoated proximal balloon portion 23a is therefore expanded first and can be brought into contact with the wall of the vessel V, before the distal coated portion 23b of the balloon 23 is expanded. This intermediate state is illustrated in FIG. 2B.

FIG. 2C shows the end state of the full expansion of the entire length of the balloon 23, including its distal coated portion 23b. The X-ray marker rings 27a, 27b and 27c in this case again mark key portions of the balloon, more specifically in this sequence: the distal balloon end, the border between the coated and uncoated portions, and the proximal balloon end. So as to be able to displace the protective sleeve from proximally to distally, a push rod or wire 29 is provided in the catheter body 21 and reaches as far as the proximal end of the balloon catheter 20, where it can be slid distally. Its distal end is fastened to the distal end 25b of the displaceable protective sleeve 25 so that it entrains the protective sleeve in the distal direction.

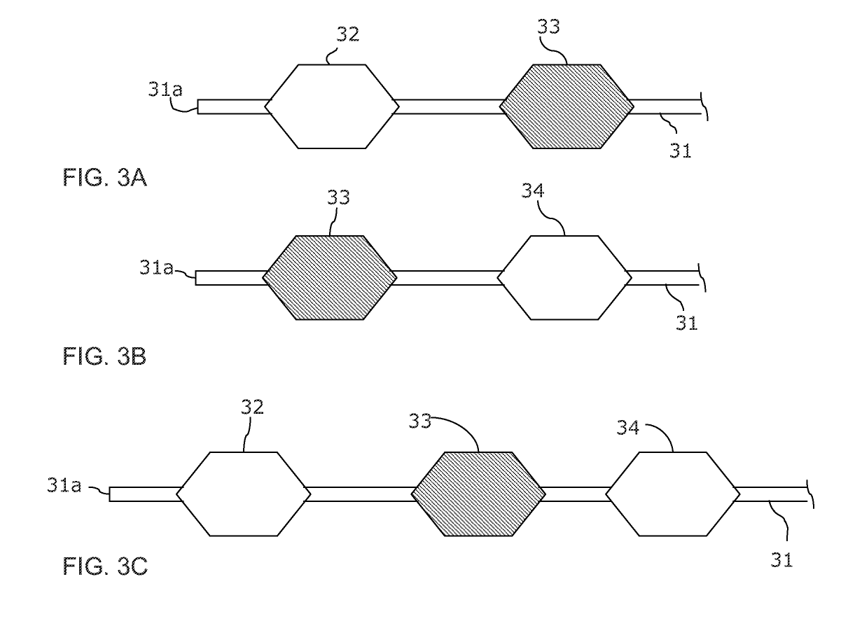

As a further embodiment FIG. 3A shows a balloon catheter with two separate balloons 32, 33 on a catheter body 31, of which the proximally arranged balloon 33 is drug-coated and the distally arranged balloon 32 by contrast is free from a drug coating (uncoated). As a further embodiment FIG. 3B shows a balloon catheter, which likewise has two balloons 33 and 34, wherein the coated balloon 33 is in this case arranged distally. FIG. 3C lastly shows a further balloon catheter, which comprises three balloons 32, 33 and 34, wherein an uncoated balloon 32 and 34 respectively are arranged distally and proximally from the coated balloon 33.

In accordance with a specific embodiment the properties of expanded PTFE (Polytetrafluoroethylene), known as e-PTFE, are utilized for forming the tip 15a. An e-PTFE tube that corresponds in terms of its outer diameter to the protecting outer tube is fastened to the outer tube an adhesively bonded connection. In contrast to other PTFE types, e-PTFE can be adhesively bonded very easily if low-viscosity adhesives are used that are capable of wetting non-polar surfaces. In this case, the microstructure of the e-PTFE is primarily mechanically anchored with the penetrating adhesive. A very reliable adhesive bond/anchoring of the e-PTFE component with the adhesive is obtained.

In an example, e-PTFE tubes by Zeuss and the cyanoacrylate adhesive Loctite 4061 are used. The e-PTFE tube is stretched by long-nose pliers to produce an improved transition from the protective tube to the tip segment. Alternatively, a rolled e-PTFE film can be used instead of an e-PTFE tube. The connection point is fixed by the cyanoacrylate adhesive. The adhesive Loctite 4061 very effectively wets the e-PTFE tube at the contact points and also has a suitable viscosity so as to infiltrate the e-PTFE tube.

The protruding region is saturated in an isopropanol/stearic acid solution so as to transfer an amount of the fatty acid, controlled by the stearic acid concentration, to the PTFE through a dipping process. This solution is stable from a temperature above the melting point of the fatty acid stearic acid used. The saturated region is then dried in hot air ≅80° C. In this case, a temperature higher than the melting point of the fatty acid should be used, since this has a high tendency for crystallization and at excessively low temperature produces a crystalline precipitate poorly connected to the e-PTFE substrate.

In this process step, the e-PTFE is infiltrated by the stearic acid and can be formed via thermal forming processes. The composite produced can then be brought via pressure into a temporary form by temperatures above the melting point of the fatty acid used.

This composite is relatively stable under pressure, but the PTFE regions delaminate under tensile force and the e-PTFE tube is exposed again. Depending on the production of the e-PTFE, it may still be deformable. The properties used primarily however are the forming of a composite that is stable under pressure and under tensile loads and that delaminates easily under tensile load in the regions “adhesively bonded” by the fatty acid. The delaminated composite has low friction, and also a specific softness depending on the formulation, which also makes this structural region of the catheter atraumatic.

Formulations are thus possible that enable the forming of the e-PTFE composite into an atraumatically shaped tip or catheter segment, which does not produce any precariously stiff and possibly sharp-edged, deformed regions after its plastic deformation. Such a catheter segment can be withdrawn without difficulty through a narrow passage, such as an introducer or a hemostatic port, even though this catheter segment will have been previously deformed to greater diameters.

Due to its recovered softness, this composite also protects, after deformation, against damage that could be caused by contact with the vessel.

The composite can be used to form a catheter tip that is pressed on internally (by a dilating balloon). In this case, the glued regions of the composite delaminate under the tensile load produced. If an e-PTFE membrane type that can be expanded yet further is used, this tip can be stretched more considerably (by approximately 4 times the diameter according to current tests). The microstructure of the e-PTFE composite also delaminates as a result of the deformation, and the membrane reassumes its original softness approximately.

In the event of deformation by an inwardly located balloon, the deformation can be determined primarily by the length of the e-PTFE tube used, such that a protective funnel is produced by the deformed tip and ends before the diameter that would produce contact with the vessel wall.

Advantageous secondary effects include:
The catheter segment can be plastically deformed. This deformation produces a soft, flexible and therefore also atraumatic structure. The flexibility of this structure produced by the reforming process makes deformation unproblematic, for example for the refraction and further handling of the catheter
The e-PTFE and also the stearic-acid-coated composite are characterized by a low-friction surface.
Minimal particle formation is detectable under microscope with the use of pure stearic acid. The fixing of the fatty acid in the PTFE structure and at the surface can be considered to be very effective. By reducing the crystallinity of this binding system (for example, in the case of stearic acid, by admixing oleic acid), requirements of particle formation and also reduction of the composite strength can be optimized in a simple form.

The embodiment of the invention is not limited to the above-described examples and highlighted features, but is also possible in a large number of modifications that lie within the scope of routine activity in the art.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A balloon catheter comprising: a drug-coated balloon having an uncoated region free from drug coating, a displaceable protective sleeve covering the drug-coated balloon and an elongate catheter body that has a distal end and a proximal end.

2. The balloon catheter as claimed in claim 1, wherein the uncoated region is a distal region of the drug-coated balloon.

3. The balloon catheter as claimed in claim 2, wherein the distal region of the drug-coated balloon has a smaller diameter than the remaining balloon and/or tapers toward its distal end.

4. The balloon catheter as claimed in claim 1, wherein the uncoated region is a proximal region of the drug-coated balloon.

5. The balloon catheter as claimed in claim 1, wherein the position of the drug-coated balloon is marked by at least one X-ray marker.

6. The balloon catheter as claimed in claim 1, wherein an end of each the drug-coated balloon and the uncoated region is marked by an X-ray marker.

7. The balloon catheter as claimed in claim 1, wherein a border of the uncoated region marked by an X-ray marker.

8. The balloon catheter as claimed in claim 1, further comprising displacement means for displacing the protective sleeve to first expose the uncoated region and sub subsequently expose the remaining drug-coated balloon.

9. The balloon catheter as claimed in claim 8, wherein the displacement means comprise a push wire or a push rod, which reaches the proximal end of the catheter body, where it is provided with an actuation device.

10. The balloon catheter as claimed in claim 1, wherein an end of the protective sleeve is expandable in a flexible manner in response to movement of the drug coated balloon out of the end of the protective sleeve.

11. The balloon catheter as claimed in claim 10, wherein the end of the protective sleeve comprises an e-PTFE composite formed into an atraumatically shaped tip.

12. A balloon for forming a balloon catheters as claimed in claim 1, wherein the uncoated region comprises an end region of the balloon.

13. A balloon catheter comprising: a drug-coated balloon, a displaceable protective sleeve covering the drug-coated balloon, an elongate catheter body that has a distal end and a proximal end, and an uncoated balloon free from the drug coating, wherein the drug-coated balloon and the uncoated balloon are apart from each other within the displaceable protective sleeve.

14. The balloon catheter as claimed in claim 13, wherein the uncoated balloon has a smaller diameter than the drug-coated balloon.

15. The balloon catheter as claimed in claim 13, wherein the drug-coated balloon and the uncoated balloon are connected to separate lumens in the catheter body in such a way that they can be dilated with a different blowing pressure.

16. The balloon catheter as claimed in claim 13, wherein the position of the drug-coated balloon is marked by at least one X-ray marker.

17. The balloon catheter as claimed in claim 13, wherein an end of each the drug-coated balloon and the uncoated balloon is marked by an X-ray marker.

18. The balloon catheter as claimed in claim 13, wherein an end of the protective sleeve is expandable in a flexible manner in response to movement of the drug coated balloon and uncoated balloon out of the end of the protective sleeve.

19. The balloon catheter as claimed in claim 18, wherein the end of the protective sleeve comprises an e-PTFE composite formed into an atraumatically shaped tip.
